(19) United States
(12) Patent Application Publication
(10) Pub. No.: US 2018/0228819 A1
(43) Pub. Date: Aug. 16, 2018

(54) COMPOSITIONS COMPRISING CHOLINE AND DERIVATIVES THEREOF, USES THEREOF AND PROCESSES FOR THEIR PREPARATION

(71) Applicant: ENZYMOTEC LTD., Kfar Baruch (IL)

(72) Inventors: Asher WIDBERG, Haifa (IL); Ran NUMA, Haifa (IL); Gai BEN-DROR, Gita (IL); Fabiana BAR-YOSEPH, Haifa (IL); Itay SHAFAT, Yokneam Illit (IL)

(73) Assignee: ENZYMOTEC LTD., Kfar Baruch (IL)

(21) Appl. No.: 15/568,465

(22) PCT Filed: Apr. 21, 2015
(86) PCT No.: PCT/IB2015/052902

§ 371 Date: Oct. 20, 2017

(51) Int. Cl. A61K 31/661; A23L 33/00; A23L 33/15; A61K 31/14; A61P 3/04

(52) U.S. Cl. CPC A61K 31/661; A23L 33/30; A23L 33/15; A23V 2002/00; A61K 31/14; A61P 3/04; A23L 33/40

(57) ABSTRACT

The invention provides compositions comprising choline and water soluble derivatives thereof, processes for their preparation and uses thereof.

### TECHNOLOGICAL HELD

The invention provides compositions comprising choline and water soluble derivatives thereof, processes for their preparation and uses thereof.

### BACKGROUND ART

References considered to be relevant as background to the presently disclosed subject matter are listed below:

1. Blusztajn J K. Choline, a vital amine. Science 1998; 281(5378)794-795.

2. Zeisel S H. Choline: an essential nutrient for humans. Nutrition 2000; 16(7-8): 669-671.

3. Food and Nutrition Board, Institute of Medicine. Choline. In: Dietary Reference Intakes for Thiamin, Riboflavin, Niacin, Vitamin B6, Vitamin B12, Pantothenic Acid, Biotin, and Choline. Washington, D.C.: National Academy Press, 1998: 390-422.

4. Holmes-McNary M Q, Cheng W L, Mar M H, Fussell S, Zeisel S H. Choline and choline esters in human and rat milk and in infant formulas. Am J Clin Nutr. 1996 Oct; 64(4): 572-6.

5. Holmes H C, Snodgrass G J, Iles R A. Changes in the choline content of human breast milk in the first 3 weeks after birth. Eur J Pediatr. 2000 Mar; 159(3): 198-204.

6. Ilcol Y O, Ozbek R, Hamurtekin E. Ulus I H. Choline status in newborns, infants, children, breast-feeding women, breast-fed infants and human breast milk J Nutr Biochem. 2005 Aug; 16(8): 489-99.

7. Kristine Y. Patterson, Seema A. Bhagwat, Juhi R. Williams, Juliette C. Howe, and Joanne M. Holden. USDA Database for the Choline Content of Comnion Foods. Release Two. Nutrient Data Laboratory Agricultural Research Service U.S. Department of Agriculture. January 2008.

8. Florian J. and Warshel. Phosphate Ester Hydrolysis in Aqueous Solution: Associative versus Dissociative Mechanisms. A. J. Phys. Chem. B. 1998, 102 (4), pp 719-734.

9. Kamerlin S C, Florian J, Warshel A. Associative versus dissociative mechanisms of phosphate monoester hydrolysis: on the interpretation of activation entropies. Chemphyschem. 2008 Aug 25; 9(12): 1767-73.

10. Vincent J B, Crowder M W, Averill B A. Hydrolysis of phosphate monoesters: a biological problem with multiple chemical solutions. Trends Biochem Sci. 1992 Mar; 17(3): 105-10.

11. U.S. Pat. No. 2864848 A1—Method of producing 1-alpha-glycerylphosphorylcholine.

12. WO 2007/010892 A1—Novel phospholipid processing agent.

13. Ozarda Y l, Cansev M, Ulus I H. Breast milk choline contents are associated with inflammatory status of breastfeeding women. J Hum Lact. 2014 May; 30(2): 161-6.

14. Ozarda Y l, Cansev M, Ulus I H. Relations of human breastmilk choline content with maternal hormonal status. Breastfeed Med. 2014 Jan-Feb; 9(1): 39-41.

15. Kanner J and Lapidot T. The stomach as a bioreactor: dietary lipid peroxidation in the gastric fluid and the effects of plant-derived antioxidants, Free Radical Biology and Medicine, Volume 31, Issue 11, 2001, Pages 1388-1395.

16. US 2003/206881 A1—Phospholipidic composition as well as the use thereof.

17. WO 98/32428 A2—Composition comprising choline and use of choline to treat endotoxic shock.

18. US 2004/043013 A1—Metabolic uncoupling therapy.

19. WO 2005/006890 A2—Foods, beverages, condiments, spices and salad dressings with specialized supplements.

Acknowledgement of the above references herein is not to be inferred as meaning that these are in any way relevant to the patentability of the presently disclosed subject matter.

### BACKGROUND

Choline is an essential dietary component and its consumption is needed to maintain health, despite the fact that mammals can synthesize it in small amounts. Water soluble choline compounds such as phosphocholine (PCh), glycerophosphocholine (GPC) and choline (free choline and choline salt), serve a number of essential biological functions including preservation of the structural integrity of cell membranes, cell signaling, nerve impulse transmission, lipid (fat) transport and metabolism, and are also a source of methyl groups.

In 1998, the Food and Nutrition Board (FNB) of the Institute of Medicine set an Adequate Intake (AI) level for choline (Table 1). The main criteria for establishing the AI for choline was the prevention of liver damage.

TABLE 1
Adequate Intake (AI) for Choline

| Population | Age | AI (mg/day) | Upper limit (g/day) |
|---|---|---|---|
| Infants | 0-6 months | 125 | Not possible to establish. |
| | | ≅18 mg/kg | Source of intake should |
| | 7-12 months | 150 | be formula and food only |
| | | ≅17 mg/kg | |
| Children | 1-3 years | 200 | 1 |
| | 4-8 years | 250 | 1 |
| | 9-13 years | 375 | 2 |
| Males | 14-18 years | 550 | 3 |
| | ≥19 | 550 | 3.5 |
| Females | 14-18 years | 400 | 3 |
| | ≥19 | 425 | 3.5 |
| Pregnancy | All ages | 450 | Age-appropriate UL |
| Lactation | All ages | 550 | Age-appropriate UL |

The current available commercial sources of GPC, PCh and choline are either naturally occurring or synthetically made. Naturally occurring sources have the significant drawback of containing highly variable and minimal levels of GPC, choline and PCh (see USDA Database for the Choline Content of Common Foods, 2008) and are thus not suitable for dietary supplementation.

Therefore, the only available sources of GPC, choline and PCh that appear in controlled, concentrated and purified form are from synthetic sources. The synthetic sources of choline, GPC and PCh are formed by chemical or enzymatic processes from different starting materials and usually involve the use of either undesirable starting materials or catalysts that may limit the use of those synthetic products for food application, especially for infant nutrition.

Beside the limitation of using those synthetic products, previous data showed that the phosphate-esters compounds—including PCh and GPC—are potentially vulnerable compounds, and thus expected to be unstable during exposure to high temperature, oxygen and water. In addition, studies have shown that metal ions neutralize the negative charge on the phosphate, making it more susceptible to nucleophilic attack. The metal ions might also be able to accelerate the rate of phosphate ester hydrolysis by any or all of the following, without wishing to be limited by the following: (a) stabilizing the leaving group (RO—) by coordination, (b) providing an effective OH— nucleophile at physiological pH and (c) organizing the reactants H₂O and ROPO₃⁻² to make the reaction effectively intramolecular. For example, during the production process, infant formulas are subjected to an environment that includes all of these problematic, risky parameters for PCh and GPC compounds—hydration, high temperature, oxygen, and metal ions. Therefore, these compounds are at increased risk of degradation if added to infant formulas.

Data collected from human milk demonstrate that the water soluble choline compounds concentrations are inconsistent in regards with the ratio between the different choline compounds. While some studies demonstrated that PCh is the major compound of choline followed by GPC, others demonstrated the opposite. Although PCh and GPC are the most prevalent choline compounds in human milk, no recommendations or regulations have been defined regarding supplementation of these water soluble choline compounds. Infant formulas contain significantly high levels of choline compared to human milk since they are usually supplemented only by choline salts—particularly choline bitartrate and choline chloride.

Choline supplementation might promote adverse effects since several studies demonstrated that choline is metabolized by gut flora, forming the metabolites: Trimethylamine (TMA) and Trimethylamine N-oxide (TMAO). Those molecules were shown to predict risk for Cardiovascular disease (CVD) in an independent large clinical cohort and to promote up-regulation of multiple macrophage scavenger receptors linked to atherosclerosis and CVD risk.

### GENERAL DESCRIPTION

The present invention discloses for the first time that certain water soluble choline compounds have an advantage over other water soluble choline compounds in improving certain parameters and conditions.

In the first aspect of the present invention there is provided a composition comprising at least one water soluble choline compound selected from a group consisting of GPC, PCh, and choline; wherein said at least one water soluble choline compound is derived from at least one natural source, and wherein the concentration of said at least one water soluble choline compound is at least 0.5% w/w of the composition.

As used herein above and below, the term “w/w percentage” or “% w/w” refers to weight percentage out of dry matter weight.

As used herein above and below the term “water soluble choline compound” refers to any choline derivative that is soluble in water, such as for example compounds including choline (including both free choline and choline salt (e.g choline chloride, choline bitartarate and choline citrate)), PCh, GPC and any derivatives thereof.

As used herein above and below the term “at least one water soluble choline compound”, refers to a single water soluble choline compound or any combination of water soluble choline compound derivatives as noted above. Therefore, at least one water soluble choline compound may refer to one water soluble choline compound being selected from choline, PCh, GPC; or to two water soluble choline compounds (choline and PCh or choline and GPC or GPC and PCh); or to three water soluble choline compounds (choline, PCh and GPC).

Throughout the invention, it is important to note that the term “composition” encompasses any type of pharmaceutical, nutraceutical, food composition or supplement for administration and metabolization by a subject that is produced by industrial means and which may at some embodiments be derived from natural sources, however is not a natural product and cannot be understood to encompass any naturally occurring composition such as for example human milk.

As noted in the first aspect of the invention the concentration of said at least one water soluble choline compound, whether it is a single compound or a combination of water soluble choline compound derivatives is at least 0.5% w/w of the composition.

In some embodiments of the invention the concentration of the at least one water soluble choline compound is at least 1% w/w of the composition, in other embodiments at least 3% w/w of the composition, in further embodiments at least 5% w/w of the composition, in yet further embodiments at least 10% w/w of the composition and in other embodiments at least 20% of the composition. In yet further embodiments at least 24% w/w of the composition and in other embodiments at least 30% of the composition

In some embodiments the water soluble choline compounds comprise between about 1% w/w to 100% w/w of said composition.

In some other embodiments, said composition comprises a combination of at least two water soluble choline compounds selected from a group consisting of GPC, PCh, choline or any combination thereof (i.e. any one of the combinations choline and GPC; choline and PCh; GPC and PCh). In other embodiments, said composition comprises, a combination of three water soluble choline compounds consisting of GPC, PCh and choline.

In some embodiments, wherein said water soluble choline compound comprises PCh, its concentration is at least 0.02% w/w of the composition. In some embodiments the concentration of PCh in a composition of the invention is at least 0.2% w/w of the composition. In some embodiments the concentration of PCh in a composition of the invention is at least 0.5% w/w of the composition, in other embodiments at least 1% w/w of the composition, in yet other embodiments at least 2% w/w of the composition and in further embodiments at least 5% w/w of the composition.

In some other embodiments, wherein said water soluble choline compound comprises GPC, its concentration is at least 0.5% w/w of the composition. In some embodiments, the concentration of GPC in a composition of the invention is at least 1% w/w of the composition, in other embodiments at least 3% w/w of the composition, in further embodiments at least 5% w/w of the composition, in other embodiments at least 10% w/w of the composition and yet further embodiments at least 20% w/w of the composition.

In further embodiments, wherein said composition comprises, as said water soluble choline compound, a combination of two or more water soluble choline compounds, wherein one of the water soluble choline compounds is GPC, said GPC comprises at least 20% w/w of said water soluble choline compound. In other embodiments, GPC comprises between about 20% w/w to about 100% w/w of the water soluble choline compound. In other embodiments, GPC comprises between about 20% w/w to about 90% w/w of the water soluble choline compound. In other embodiments, GPC comprises between about 20% w/w to about 80% w/w of the water soluble choline compound. In other embodiments, GPC comprises between about 20% w/w to about 70% w/w of the water soluble choline compound. In other embodiments, GPC comprises between about 30% w/w to about 60% w/w of the water soluble choline compound and in yet further embodiments between about 40% w/w to about 50% w/w of the water soluble choline compound. In other embodiments, GPC comprises about 85% of the water soluble choline compound.

In some embodiments, wherein said composition comprises, as said water soluble choline compound, a combination of PCh and choline (in some other embodiments GPC, PCh and choline together). PCh and choline together constitute at least 1% w/w of the water soluble choline compound. In further embodiments, PCh and choline comprise between about 1% w/w to about 60% w/w of the water soluble choline compound. In other embodiments, PCh and choline comprise between about 10% w/w to about 50% w/w of the water soluble choline compound and in yet further embodiments between about 20% w/w to about 40% w/w of the water soluble choline compound.

In some embodiments, wherein said composition comprises, as said water soluble choline compound, a combination of GPC and PCh and optionally choline or a combination of GPC and choline and optionally PCh, PCh and choline together constitute at least 1% w/w of the water soluble choline compound. In further embodiments, PCh and choline comprise between about 1% w/w to about 60% w/w of the water soluble choline compound. In other embodiments, PCh and choline comprises between about 10% w/w to about 50% w/w of the water soluble choline compound and in yet further embodiments between about 20% w/w to about 40% w/w of the water soluble choline compound.

Throughout the invention, when referring to the fact that the at least one water soluble choline compound is “derived from natural source” it should be understood to encompass that said water soluble choline compound originated from a natural source, i.e. not a synthetic source. In some embodiments, the at least one natural source is selected from a group consisting of vegetable source, mammalian milk, animal source, egg, marine source, microorganism or aquaculture organisms and any combination thereof.

In some embodiments the natural source comprises mammalian milk (bovine milk, goat milk, sheep milk, buffalo milk and the like); in some embodiments the natural source comprises bovine milk.

Throughout the invention, the term “natural source” may also include any common and known product or food derived from the source (e.g. whey protein derived from bovine milk, skimmed bovine milk powder etc.). By common foods it is meant materials that are commonly eaten as foodstuffs. However, such common foods may optionally contain other substances added to them during preparation. For example, whey protein may contain higher levels of NaCl added during the cheese preparation process.

In some embodiments, said composition comprises at most 1 ppm of at least one of TMA, TMAO, ethylene oxide, glycidol and any combination thereof. In other embodiments said composition comprises at most 10 ppm of at least one of TMA, TMAO, ethylene oxide, glycidol and any combination thereof. In some embodiments said composition comprises at most 20 ppm of at least one of TMA, TMAO, ethylene oxide, glycidol and any combination thereof. In some embodiments said composition comprises at most 30 ppm of at least one of TMA, TMAO, ethylene oxide, glycidol and any combination thereof and in yet further embodiments at most 50 ppm.

Without wishing to be bound by any theory, molecules such as TMA and TMAO were shown to predict risk for CVD and to promote up-regulation of multiple macrophage scavenger receptors linked to atherosclerosis and CVD risk. Thus, the compositions of the invention may have beneficial effect in this respect e.g., may be used in the treatment or prevention of such diseases or reduce the risk of such diseases in a subject administered with same.

Thus, the present invention, in at least some embodiments, overcomes the drawbacks of the prior art by providing compositions containing concentrated water soluble choline compounds that are purified from natural, non-synthetic sources and are therefore free of precursors used for synthetic preparations (e.g. ethylene oxide, glycidol, TMA, etc.) and are therefore also free from substances required to synthesize such preparations, such as catalysts (either chemical or enzymatic) that may create harmful by-products or remain in residual amounts in the final product.

It is noted that the embodiments detailed herein above in connection with the first aspect of the invention are also relevant to the further aspects detailed herein below which refer to methods comprising administering said composition and the various uses of said composition.

In a further aspect the invention provides a process for the preparation of a composition comprising at least one water soluble choline compound; wherein said at least one water soluble choline compound is derived from at least one natural source, and wherein said concentration of said at least one water soluble choline compound is at least 0.5% w/w of the composition; said process comprising the steps of:

(i) providing at least one natural source of choline;

(ii) purifying said at least one natural source of choline.

Existing natural sources of water soluble choline compounds contain very low and highly variable concentrations of the compounds. Therefore, it is not commercially practical to use them for administration of water soluble choline compounds.

The inventors of the present invention have demonstrated that technologies intended for removal of contaminants that are a minority portion of the mixture (for example, less than 50% of the mixture) are also efficient for purifying those substances from very low residual levels, removing above 50% of the starting material, preferably above 80% or even 90% of the mixture.

In some embodiments of a process of the invention said at least one natural source of choline contains less than 0.5% w/w GPC, 0.5% w/w PCh and/or 0.5% w/w choline. In further embodiment of a process of the invention said at least one natural source of choline contains at most 1% w/w GPC, 0.5% w/w PCh and/or 0.5% w/w choline. In further embodiments, said at least one natural source of choline contains at most 0.4% w/w GPC, 0.4% w/w PCh and/or 0.4% w/w choline. In other embodiments, said at least one natural source of choline contains at most 0.3% w/w GPC, 0.3% w/w PCh and/or 0.3% w/w choline. In other embodiments, said at least one natural source of choline contains at most 0.2% w/w GPC, 0.2% w/w PCh and/or 0.2% w/w choline.

In further embodiments of a process of the invention said purification include, but are not limited to, extraction, crystallization, chromatography, ion exchange purification, membrane purification, ultra filtration, nano filtration, micro filtration, electrodialysis or water washes.

In another embodiment a process of the invention further comprises the step of (iii) extracting said natural source with an organic solvent comprising an alcohol of 1 to 4 carbon atoms.

In another embodiment the process result in a composition with controllable concentration of said water soluble choline compound. By “contrallable” it is meant that the concentration may be increased or decreased, for example optionally according to the needs of the composition or its final application (such as ingestion by a subject).

The invention further provides various uses of the aforementioned composition (referred to herein above as the first aspect of the invention) as well as various methods utilizing same.

Accordingly, in another one of its aspects the present invention provides a composition comprising at least one water soluble choline compound selected from a group consisting of GPC, PCh and choline; wherein said at least one water soluble choline compound is derived from mammalian milk, and wherein the concentration of said at least one water soluble choline compound is at least 0.5% w/w of the composition, for use in one or more of:

(i) optimizing, improving, promoting, increasing or maintaining plasma levels of one or more of ferritin, iron, growth hormone, insulin-like growth factor (IGF) (e.g., IGF 1), insulin-like growth factor binding protein (IGFBP) (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In yet another one of its aspects the present invention provides a composition comprising at least one water soluble choline compound selected from a group consisting of GPC, PCh and choline; wherein said at least one water soluble choline compound is derived from mammalian milk, and wherein the concentration of said at least one water soluble choline compound is at least 0.5% w/w of the composition, for use in a method for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in a further one of its aspects the present invention provides a composition comprising at least one water soluble choline compound selected from a group consisting of GPC, PCh and choline; wherein said at least one water soluble choline compound is derived from at least one natural source, and wherein the concentration of said at least one water soluble choline compound is at least 0.5% w/w of the composition, for use in one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In yet a further one of its aspects the present invention provides a composition comprising at least one water soluble choline compound selected from a group consisting of GPC, PCh and choline; wherein said at least one water soluble choline compound is derived from at least one natural source, and wherein the concentration of said at least one water soluble choline compound is al least 0.5% w/w of the composition, for use, in a method for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g,, IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in a further one of its aspects the present invention provides a composition comprising at least one water soluble choline compound selected from a group consisting of GPC, PCh and choline; wherein said at least one water soluble choline compound is derived from at least one natural source, and wherein the concentration of said at least one water soluble choline compound is at least 0.5% w/w of the composition, wherein said composition is for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in another one of its aspects the present invention provides a composition comprising at least one water soluble choline compound selected from a group consisting of GPC, PCh and choline, wherein said at least one water soluble choline compound is derived from at least one natural source, and wherein the concentration of said at least one water soluble choline compound is at least 0.5% w/w of the composition, for use in the manufacture of a pharmaceutical composition, a dietary supplement, a medical food, a nutritional or a neutraceutical composition, for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In a further one of its aspects the present invention provides a method comprising administering a composition, wherein the composition comprises at least one water soluble choline compound selected from a group consisting of GPC, PCh and choline, wherein said at least one water soluble choline compound is derived from at least one natural source, and wherein the concentration of said at least one water soluble choline compound is at least 0.5% w/w of the composition, the method being for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In some embodiments of the invention the at least one natural source is mammalian milk.

In some embodiments of the invention the at least one water soluble choline compound is any one of GPC, PCh and choline.

In some embodiments of the invention the PCh comprises at least 0.02% w/w of the composition.

In some embodiments of the invention the GPC comprises at least 0.5% w/w of the composition.

In some embodiments of the invention the GPC comprises between about 20% w/w to about 100% w/w of the at least one water soluble choline compound.

In some embodiments of the invention the GPC comprises between about 20% w/w to about 90% w/w of the at least one water soluble choline compound.

In some embodiments of the invention the GPC comprises between about 20% w/w to about 80% w/w of the at least one water soluble choline compound.

In some embodiments of the invention the GPC comprises between about 20% w/w to about 70% w/w of the at least one water soluble choline compound.

In some embodiments of the invention the GPC comprises between about 85% w/w of the at least one water soluble choline compound.

In some embodiments of the invention the PCh and choline comprise between about 1% w/w to about 60% w/w of at least one water soluble choline compound.

In some embodiments of the invention the composition comprises at most 50 ppm of at least one of TMA, TMAO, ethylene oxide, glycidol or any combination of the same.

In some embodiments of the invention the cholesterol is selected from a group consisting of total cholesterol, LDL cholesterol, VLDL cholesterol, non-HDL cholesterol, HDL cholesterol and a combination of the same.

In some embodiments of the invention the subject is an infant.

In some embodiments of the invention the subject is an adult.

In some embodiments of the invention the subject is a pregnant or lactating woman.

In another one of its aspects the invention provides a composition comprising at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds.

It is noted that in case the composition does not include one of PCh or choline their concentration is null. Thus, in some embodiments, the term “PCh and choline” refer to the two water soluble choline compounds, in other embodiments in case PCh is not part of the water soluble choline compounds of the composition the term relates only to choline; in yet further embodiments in case choline is not part of the water soluble choline compounds of the composition the term relates only to PCh.

In some embodiments, the composition comprises GPC, PCh and choline.

In some embodiments, said water soluble choline compounds comprise between about 1% w/w to 100% w/w of said composition. In other embodiments, said water soluble choline compounds comprise at least 1% w/w of the composition. In other embodiments said water soluble choline compound comprise at least 3% w/w of the composition, in further embodiments at least 5% w/w of the composition, in yet further embodiments at least 10% w/w of the composition and in other embodiments at east 20% of the composition.

In some embodiments, PCh concentration is at least 0.2% w/w of the composition. In some embodiments the concentration of PCh in a composition of the invention is at least 0.5% w/w of the composition, in other embodiments at least 1% w/w of the composition, in yet other embodiments at least 2% w/w of the composition and in further embodiments at least 5% w/w of the composition.

In some other embodiments, GPC concentration is at least 0.5% w/w of the composition. In some embodiments, the concentration of GPC in a composition of the invention is at least 1% w/w of the composition, in other embodiments at least 3% w/w of the composition, in further embodiments at least 5% w/w of the composition, in other embodiments at least 10% w/w of the composition and yet further embodiments at least 20% w/w of the composition.

In other embodiments, said GPC comprises at least 20% w/w of said water soluble choline compounds. In other embodiments, said GPC comprises between 20% w/w to about 100% w/w of said water soluble choline compounds. In other embodiments, said GPC comprises between 20% w/w to about 90% w/w of said water soluble choline compounds. In other embodiments, said GPC comprises between 20% w/w to about 80% w/w of said water soluble choline compounds. In other embodiments, said GPC comprises between 20% w/w to about 70% w/w of said water soluble choline compounds. In other embodiments, said GPC comprises between about 30% w/w to about 60% w/w of the water soluble choline compound and in yet further embodiments between about 40% w/w to about 50% w/w of the water soluble choline compound. In some embodiments of the invention the GPC comprises about 85% w/w of the at least one water soluble choline compound.

In some embodiments, said PCh comprises between about 1% w/w to 70% w/w of said water soluble choline compounds. In other embodiments, said PCh comprises between about 20% w/w to about 60% w/w of the water soluble choline compound and in yet further embodiments between about 30% w/w to about 40% w/w of the water soluble choline compound.

In other embodiments, said choline comprises between about 1% w/w to 25% w/w of said water soluble choline compounds. In other embodiments, said choline comprises between about 5% w/w to about 20% w/w of the water soluble choline compound and in yet further embodiments between about 10% w/w to about 15% w/w of the water soluble choline compound.

It should be understood that under this composition aspect, said water soluble choline compounds are derived from any available source of choline or its components. In some embodiments, at least one of said GPC, PCh or choline is derived from a natural source. In other embodiments, said at least one of said GPC, PCh or choline is derived from a synthetic source.

In some embodiments, said composition comprises at most 1 ppm of at least one of TMA, TMAO, ethylene oxide, glycidol and any combination thereof. In other embodiments, said composition comprises at most 10 ppm of at least one of TMA, TMAO, ethylene oxide, glycidol and any combination thereof. In other embodiments said composition comprises at most 20 ppm of at least one of TMA, TMAO, ethylene oxide, glycidol and any combination thereof. In some embodiments said composition comprises at most 30 ppm of at least one of TMA, TMAO, ethylene oxide, glycidol and any combination thereof and in yet further embodiments at most 50 ppm.

The invention further provides various uses of said composition as well as various methods utilizing same as detailed herein below.

It is noted that the embodiments detailed herein above in connection with said composition of the invention are also relevant to the further aspects detailed herein below which refer to methods comprising administering said composition and various uses of said composition.

Accordingly, in another one of its aspects the present invention provides a composition comprising at least two water soluble choline compounds selected from a group consisting of GPC, PCh and choline; wherein said at least two water soluble choline compounds comprise at least 0.5% w/w of the composition; wherein PCh and choline together comprise at least 1% w/w of said water soluble choline compounds; and wherein PCh comprises between about 1% w/w to 70% w/w of said water soluble choline compounds, for use in one or more of:

(i) optimizing, improving, promoting, increasing or maintaining plasma levels of one or more of ferritin, iron, growth hormone, insulin-like growth factor IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In yet another one of s aspects the present invention provides in another one of its aspects the present invention provides a composition comprising at least two water soluble choline compounds selected from a group consisting of GPC, PCh and choline; wherein said at least two water soluble choline compounds comprise at least 0.5% w/w of the composition; wherein PCh and choline together comprise at least 1% w/w of said water soluble choline compounds; and wherein PCh comprises between about 1% w/w to 70% w/w of said water soluble choline compounds, for use in a method for one or more of:

(i) optimizing, improving, promoting, increasing r maintaining plasma levels one or more of ferritin, iron, growth hormone, insulin-like growth factor (e.g., IGF insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in a further one of its aspects the present invention provides a composition comprising at least two water soluble choline compounds selected from a group consisting of GPC, PCh and choline; wherein said at least two water soluble choline compounds comprise at least 0.5% w/w of the composition; and wherein PCh and choline comprise together at least 1% w/w of said water soluble choline compounds, for use in one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in a further one of its aspects the present invention provides a composition comprising at least two water soluble choline compounds selected from a group consisting of GPC, PCh and choline; wherein said at least two water soluble choline compounds comprise at least 0.5% w/w of the composition; and wherein PCh and choline comprise together at least 1% w/w of said water soluble choline compounds, for use in a method for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in a further one of its aspects the present invention provides a composition comprising at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds, for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

Yet, in another one of its aspects the present invention provides a composition comprising at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds, for use in the manufacture of a pharmaceutical composition, a dietary supplement, a medical food, a nutritional or a nutraceutical composition, for one or more of:

(i) optimizing, improving, promoting, increasing or maintaining plasma levels of one or more of ferritin, iron, growth hormone, insulin-like growth factor (e.g,, IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In a further one of its aspects the present invention provides a method comprising administering a composition, wherein the composition comprises at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds, the method being for one or more of:

(i) optimizing, improving, promoting, increasing or maintaining plasma levels of one or more of ferritin, iron, growth hormone, insulin-like growth factor (e.g., IGF 1), insulin-like growth factor binding protein (e.g., IGFBP 3) or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same.

In some embodiments of one or more aspects of the invention the PCh comprises between about 1% w/w to 70% w/w of said water soluble choline compounds in said composition of the invention.

In some embodiments of one or more aspects of the invention the at least two water soluble choline compounds comprise at least 1% w/w of the composition of the invention.

In some embodiments of one or more aspects of the invention said composition comprises GPC, PCh and choline.

In some embodiments of the invention the GPC comprises between 20% w/w to about 100% w/w of said water soluble choline compounds in said composition of the invention. At times between 20% w/w to about 90% w/w, even at times between 20% w/w to about 80% w/w, and even at times between 20% w/w to about 70% w/w. In some embodiments of the invention the GPC comprises 85% w/w of said water soluble choline compounds in the composition of the invention.

In some embodiments of one or more aspects of the invention the PCh comprises between 1% w/w to about 70% w/w of said water soluble choline compounds.

In some embodiments of one or more aspects of the invention the choline comprises between about 1% w/w to 25% w/w of said water soluble choline compounds in said composition of the invention.

In some embodiments of one or more aspects of the invention the at least one of said GPC, PCh or choline is derived from a natural source.

In some embodiments of the invention the natural source is mammalian milk.

In some embodiments of the invention at least one of said GPC, PCh or choline is derived from a synthetic source.

In some embodiments of one or more aspects of the invention the molar concentration of PCh in said composition of the invention is greater than the molar concentration of GPC.

In a further aspect the invention provides a composition comprising GPC and PCh; wherein the molar concentration of PCh is greater than the molar concentration of GPC.

In a further aspect the invention provides a composition comprising GPC and PCh; wherein the molar concentration of PCh equals the molar concentration of GPC.

In some embodiments, GPC and PCh comprise at least 0.05% of the composition.

In some embodiments, said GPC and PCh comprise at least 0.1% w/w of the composition; in other embodiments at least 0.5% w/w of the composition; in further embodiments at least 1% w/w of the composition, in other embodiments at least w/w of the composition and in further embodiments at least 5% w/w of the composition. In other embodiments at least 10% w/w of the composition and in further embodiments at least 20% w/w of the composition.

In further embodiments, said composition further comprises choline wherein GPC, PCh and choline comprise at least 0.05% w/w of the composition.

In some embodiments, said GPC, PCh and choline comprise at least 0.1% w/w of the composition; in other embodiments at least 0.5% w/w of the composition; in further embodiments at least 1% w/w of the composition, in other embodiments at least 2% w/w of the composition and in further embodiments at least 5% w/w of the composition. In other embodiments at least 10% w/w of the composition and in further embodiments at least 20% w/w of the composition.

In some further embodiments, said composition is capable of being chemically stable at storage temperatures of 20-30° C. for at least 12 months. In other embodiments said composition is capable of being chemically stable at storage temperatures of 23-27° C. for at least 12 months. In other embodiments said composition is capable of being chemically stable at storage temperatures of 25° C. for at least 12 months.

In another embodiment GPC and PCh levels are stable at up to 42° C. for at least 3 months with not more than 20% degradation. In a further embodiment GPC and PCh levels are stable at 38-42° C. for at least 3 months. Optionally the levels are stable for at least 4 months. Optionally the levels are stable for up to 6 months.

In a further embodiment, said stability refers to a degradation level of at least one water soluble choline compound of less than 20% w/w. In other embodiments said composition has a degradation level of at least one water soluble choline compound of less than 15% w/w. In further embodiments said composition has a degradation level of at least one water soluble choline compound of less than 10% w/w and in yet another embodiment of less than 5%.

In a further embodiment, said stability refers to a degradation level of GPC and/or PCh of 20% w/w or less, In other embodiments said composition has a degradation level of GPC and/or PCh of 15% w/w or less. In further embodiments said composition has a degradation level of GPC and/or PCh of 10% w/w or less and in yet another embodiment of 5% or less, preferably 1% or less.

In some further embodiments, the molar concentration of GPC is greater than the molar concentration of choline (i.e. [PCh]>[GPC]>[choline]). In other embodiments, the molar concentration of choline is greater than the molar concentration of GPC and lower than the molar concentration of PCh (i.e. [PCh]>[choline]>[GPC]. In yet other embodiments, the molar concentration of choline is greater than the molar concentration of PCh (i.e. [choline]>[PCh]>[GPC]). In further embodiments, the molar concentration of PCh is greater than the molar concentration of choline.

In further embodiments of the invention, the weight ratio of PCh to GPC is at least about 0.70. In some embodiments, the weight ratio of PCh to GPC is at least about 0.8. In other embodiments, the weight ratio of PCh to GPC is at least about 1. In further embodiments, the weight ratio of PCh to GPC is at least about 1.1 or 1.2. In other embodiments at least 1.5, in other embodiments at least 2 and in yet another embodiment at least about 3.

In some embodiments, the composition of the invention further comprises betaine.

In other embodiments, the composition of the invention is formulated to a pharmaceutical, a dietary supplement, a medical food a nutritional or a neutraceutical composition.

In another aspect the invention provides a composition of the invention as described hereinabove for use in the preparation of a pharmaceutical, a dietary supplement, a medical food, a nutritional or a neutraceutical composition.

In another aspect the invention provides a pharmaceutical, a dietary supplement, a medical food a nutritional or a nutraceutical composition comprising a composition of the invention.

A medical food as used herein is any food product that has been formulated and intended for the dietary management of a subject suffering from a disease, disorder or condition that has distinctive nutritional needs which are difficult to meet with normal diet alone.

In certain other non-limiting embodiments of the present invention, the composition of the invention is formulated into a food product or a dietary supplement selected from a biscuit, pastry, cake, bread, cereal, bar, snack, pill, tablet, pellets, dragees, capsule, soft gel, syrup, infant formula, baby formula, toddler food, adult formula, medical nutrition product, candy, gummy, or confectionary.

Pharmaceutical compositions and dietary supplements suitable for oral administration may be presented as discrete dosage units such as pills, tablets, capsules, or as a powder or granules, or as a solution or suspension.

The invention further provides a formula comprising a composition of the invention. In some embodiments, said formula further comprising at least one of a physiologically acceptable lipid, protein, carbohydrate, vitamin, mineral, amino acid, nucleotide and active or non-active additive. In some embodiments said formula is an infant formula, In some embodiments said formula is a follow on formula (for babies over 6 months) or a toddler formula. In some embodiments said formula is a child formula. In some embodiments said formula is an adult formula.

In another aspect the invention provides a method for producing pharmaceutical or nutritional composition as described herein comprising a spray drying process wherein the water soluble choline compounds maintain their stability.

According to some embodiments of the present invention, during formula production, the compositions of the invention are added with all other minerals and vitamins prior to homogenization and spray drying or other methods.

In some embodiments, said lipid comprises one or more of palm and palm kernel oils, soybean oil, palm olein, coconut oil, canola oil, olive oil, cottonseed oils, medium chain triglyceride (MCT) oil, sunflower oil, high oleic sunflower oil, safflower oil, high oleic safflower oil, algal oil, marine oils and combinations thereof; wherein said protein comprises hydrolyzed, partially hydrolyzed, non-hydrolyzed or intact proteins, and any combinations thereof; wherein amino acids are selected from the group consisting of alanine, arginine, asparagine, camitine, aspartic acid, cystine, glutamic acid, glutamine, glycine, histidine, isoleucine, leucine, lysine, methionine, phenylalanine, proline, serine, taurine, threonine, tryptophan, taurine, tyrosine, valine, and combinations thereof; wherein the carbohydrate comprises one or more of hydrolyzed, intact, naturally and/or chemically modified cornstarch, maltodextrin, glucose polymers, sucrose, corn syrup, corn syrup solids, rice or potato derived carbohydrate, glucose, fructose, lactose, high fructose corn syrup and indigestible oligosaccharides such as fructooligosaccharides (FOS), galactooligosaccharides (GOS), and combinations thereof.

In another aspect the invention provides a method for optimizing, improving, promoting or maintaining the development of phospholipid synthesis or lipoprotein synthesis in a subject comprising administering a composition of the invention as described herein.

In another aspect of the invention the composition of the invention as described herein is for (or used for, or used in a method for) optimizing, improving, promoting or maintaining the development of phospholipid synthesis or lipoprotein synthesis in a subject.

In another aspect the invention provides a method for improving, promoting or maintaining proper sulphur amino acid metabolism, in a subject comprising administering a composition of the invention as described herein.

In another aspect of the invention the composition of the invention as described herein is for (or used for, or used in a method for) improving, promoting or maintaining proper sulphur amino acid metabolism in a subject.

In another aspect the invention provides a method for optimizing, improving, promoting or maintaining choline plasma levels in a subject comprising administering a composition of the invention as described herein.

In another aspect of the invention the composition of the invention as described herein is for (or used for, or used in a method for) optimizing, improving, promoting or maintaining choline plasma levels in a subject.

In another aspect the invention provides a method for optimizing, improving, promoting or maintaining one or more of choline, phosphatidylcholine, GPC and PCh plasma levels in a subject comprising administering a composition of the invention as described herein.

In another aspect of the invention the composition of the invention as described herein is for (or used for, or used in a method for) optimizing, improving, promoting or maintaining one or more of choline, phosphatidylcholine, GPC and PCh plasma levels in a subject.

In another aspect the invention provides a method for optimizing, improving, promoting or maintaining osmo-regulation in a subject comprising administering a composition of the, invention as described herein.

In another aspect of the invention the composition of the invention as described herein is for (or used for, or used in a method for) optimizing, improving, promoting or maintaining osmo-regulation in a subject.

In another aspect the invention provides a method for optimizing, improving, promoting, increasing or maintaining plasma levels of one or both of growth hormone or ketone bodies in a subject comprising administering a composition of the invention.

In another aspect of the invention the composition of the invention as described herein is for (or used for, or used in a method for) optimizing, improving, promoting, increasing or maintaining plasma levels of one or both of growth hormone or ketone bodies in a subject.

In another aspect the invention provides a method for optimizing, improving, promoting, increasing or maintaining plasma levels of one or more of growth hormone, insulin-like growth factor (e.g., IGF 1), IGF binding protein (e.g., IGFBP3) or ketone bodies in a subject comprising administering a composition of the invention.

In another aspect of the invention the composition of the invention as described herein is for (or used for, or used in a method for) optimizing, improving, promoting, increasing or maintaining plasma levels of one or more of growth hormone, insulin-like growth factor, (e.g., IGF 1), IGF binding protein (e.g., IGFBP3) or ketone bodies in a subject.

In another aspect the invention provides a method for optimizing, improving, promoting, increasing or maintaining plasma levels of one or more of ferritin, iron, growth hormone, insulin-like growth factor (e.g., IGF 1), IGF binding protein (e.g., IGFBP3) or ketone bodies in a subject comprising administering a composition of the invention.

In another aspect of the invention the composition of the invention as described herein is for (or used for, or used in a method for) optimizing, improving, promoting, increasing or maintaining plasma levels of one or more of ferritin, iron, growth hormone, insulin-like growth factor. (e.g., IGF 1), IGF binding protein (e.g., IGFBP3) or ketone bodies in a subject.

In another aspect the invention provides a method for optimizing, improving, promoting, increasing or maintaining plasma levels of one or both of insulin-like growth factor (e.g, IGF 1) or IGF binding protein (e.g., IGFBP3) in a subject comprising administering a composition of the invention.

In another aspect of the invention the composition of the invention as described herein is for (or used for, or used in a method for) optimizing, improving, promoting, increasing or maintaining plasma levels of one or both of insulin-like growth factor (e.g., IGF 1) or IGF binding protein (e.g., IGFBP3) in a subject.

In another aspect the invention provides a method for optimizing, improving, promoting, increasing or maintaining plasma levels of insulin-like growth factor (e.g., IGF 1) in a subject comprising administering a composition of the invention.

In another aspect of the invention e composition of the invention as described herein is for (or used for, or used in a method for) optimizing, improving, promoting, increasing or maintaining plasma levels of insulin-like growth factor (e.g., IGF 1) in a subject.

In another aspect the invention provides a method for optimizing, improving, promoting, increasing or maintaining plasma levels of insulin-like growth factor binding protein (e.g., IGFBP3) in a subject comprising administering a composition of the invention.

In another aspect of the invention the composition of the invention as described herein is for (or used for, or used in a method for) optimizing, improving, promoting, increasing or maintaining plasma levels of insulin-like growth factor binding protein IGFBP3) in a subject.

In some embodiments of one or more aspects of the invention the insulin-like growth factor is insulin-like growth factor 1.

In some embodiments of one or more aspects of the invention the insulin-like growth factor binding protein is insulin-like growth factor binding protein 3.

In another aspect the invention provides a method for optimizing, improving, promoting or maintaining one or both of ferritin and iron plasma levels in a subject comprising administering a composition of the invention.

In another aspect of the invention the composition of the invention as described herein is for (or used for, or used in a method for) optimizing, improving, promoting or maintaining one or both of ferritin and iron plasma levels in a subject.

In another aspect the invention provides a method for optimizing, improving, promoting, increasing or maintaining plasma levels of ferritin in a subject comprising administering a composition of the invention.

In another aspect of the invention the composition of the invention as described herein is for (or used for, or used in a method for) optimizing, improving, promoting, increasing or maintaining plasma levels of ferritin in a subject.

In another aspect the invention provides a method for optimizing, improving, promoting, increasing or maintaining plasma levels of iron in a subject comprising administering a composition of the invention.

In another aspect of the invention the composition of the invention as described herein is for (or used for, or used in a method for) optimizing, improving, promoting, increasing or maintaining plasma levels of iron in a subject.

In another aspect the invention provides a method for optimizing, improving, promoting or maintaining intestinal absorption of minerals, trace elements, metals or vitamins in a subject comprising administering a composition of the invention.

In another aspect of the invention the composition of the invention as described herein is for (or used for, or used in a method for) optimizing, improving, promoting or maintaining intestinal absorption of minerals, trace elements, metals or vitamins in a subject.

In another aspect the invention provides a method for optimizing, improving, promoting or maintaining one or more of plasma lipid profile, triglyceride levels, total cholesterol levels, low-density lipoprotein (LDL) cholesterol levels, very-low-density lipoprotein (VLDL) cholesterol levels, non-HDL cholesterol levels and high-density lipoprotein (HDL) cholesterol levels in a subject comprising administering a composition of the invention.

In another aspect of the invention the composition of the invention as described herein is for (or used for, or used in a method for) optimizing, improving, promoting or maintaining one or more of plasma lipid profile, triglyceride levels, total cholesterol levels, low-density lipoprotein (LDL) cholesterol levels, very-low-density lipoprotein (VLDL) cholesterol levels, non-HDL cholesterol levels and high-density lipoprotein (HDL) cholesterol levels in a subject.

In another aspect the invention provides a method for optimizing, improving, promoting or maintaining choline/betaine ratio in the plasma of a subject comprising administering a composition of the invention,

In another aspect of the invention the composition of the invention as described herein is for (or used for, or used in a method for) optimizing, improving, promoting or maintaining choline/betaine ratio in the plasma of a subject.

In another aspect the invention provides a method for optimizing, improving, reducing or maintaining dimethylglycine (DMG) levels in a subject comprising administering a composition of the invention.

In another aspect of the invention the composition of the invention as described herein is for (or used for, or used in a method for) optimizing, improving, reducing or maintaining dimethylglycine (DMG) levels in a subject.

In another aspect the invention provides a method for optimizing, improving, promoting or maintaining S-Adenosyl methionine (SAM) levels in a subject comprising administering a composition of the invention.

In another aspect of the invention the composition of the invention as described herein is for (or used for, or used in a method for) optimizing, improving, promoting or maintaining SAM levels in a subject.

In another aspect the invention provides a method for optimizing, improving, reducing or maintaining S-Adenosyl-L-homocysteine (SAH) levels in a subject comprising administering a composition of the invention.

In another aspect of the invention the composition of the invention as described herein is for (or used for, or used in a method for) optimizing, improving, reducing or maintaining; SAH levels in a subject.

In another aspect the invention provides a method for optimizing, improving, promoting or maintaining levels of one or more of cysteine, homocysteine and methionine in a subject comprising administering a composition of the invention.

In another aspect of the invention the composition of the invention as described herein is for (or used for, or used in a method for) optimizing, improving, promoting or maintaining levels of one or more of cysteine, homocysteine and methionine in a subject.

In another aspect the invention provides a method for optimizing, improving, promoting or maintaining amino acid profile in a subject comprising administering a composition of the invention.

In another aspect of the invention the composition of the invention as described herein is for (or used for, or used in a method for) optimizing, improving, promoting or maintaining amino acid profile in a subject.

In another aspect the invention provides a method for optimizing, improving, promoting or maintaining cognitive functions in a subject comprising administering a composition of the invention.

In another aspect of the invention the composition of the invention as described herein is for (or used for, or used in a method for) optimizing, improving, promoting or maintaining cognitive functions in a subject.

In another aspect the invention provides a method for optimizing, improving, promoting or maintaining levels of mineral or metals absorption in a subject comprising administering a composition of the invention.

In another aspect of the invention the composition of the invention as described herein is for (or used for, or used in a method for) optimizing, improving, promoting or maintaining; levels of mineral or metals absorption in a subject.

In another aspect the invention provides a method for optimizing, improving, promoting or maintaining gut flora balance in a subject comprising administering a composition of the invention.

In another aspect of the invention the composition of the invention as described herein is for (or used for, or used in a method for) optimizing, improving, promoting or maintaining gut flora balance in a subject.

In another aspect the invention provides a method for promoting optimal growth, improving statural growth or a combination of the same in a subject, comprising administering a composition of the invention.

In another aspect of the invention the composition of the invention as described herein is for (or used for, or used in a method for) promoting optimal growth, improving statural growth or a combination of the same in a subject.

In another aspect the invention provides a method for promoting optimal growth in a subject comprising administering a composition of the invention.

In another aspect of the invention the composition of the invention as described herein is for (or used for, or used in a method for) promoting optimal growth in a subject.

In another aspect the invention provides a method for improving statural growth comprising administering a composition of the invention.

In another aspect of the invention the composition of the invention as described herein is for (or used for, or used in a method for) improving statural growth in a subject.

As used herein, “optimal growth” is envisaged as a growth in which growth rate and composition of gained weight mimic that of breast-fed infants.

As used herein, “statural growth” is envisaged as growth in infant length, contrary to growth in adiposity, leading to lesser future gains of adiposity and in un-healthy body mass index (BMI).

In another aspect the invention provides a method for preventing, delaying or reducing obesity, reducing body mass index or a combination of the same in a subject comprising administering a composition of the invention.

In another aspect of the invention the composition of the invention as described herein is for (or used for, or used in a method for) preventing, delaying or reducing obesity, reducing body mass index or a combination of the same in a subject.

In another aspect the invention provides a method for preventing, delaying or reducing obesity in a subject comprising administering a composition of the invention.

In another aspect of the invention the composition of the invention as described herein is for (or used for, or used in a method for) preventing, delaying or reducing obesity in a subject.

In another aspect the invention provides a method for reducing body mass index in a subject comprising administering a composition of the invention.

In another aspect of the invention the composition of the invention as described herein is for (or used for, or used in a method for) reducing body mass index in a subject.

In a further aspect the invention provides a method for preventing or treating or improving or reducing symptoms of one or more of: Neurodegenerative diseases, Alzheimer's disease, Parkinson's disease, Amyotrophic lateral sclerosis (ALS), dementia, stroke, cognitive decline, chemotherapy-related cognitive decline, malnutrition or unbalanced nutrition, insufficient oral food intake, liver disease, liver dysfunction, alcoholic liver disease or renal dysfunction, comprising administering a composition of the invention.

In a further aspect the invention provides a method for preventing or treating or improving or reducing symptoms of one or more of: Neurodegenerative diseases, Alzheimer's disease, Parkinson's disease, Amyotrophic lateral sclerosis (ALS), dementia, stroke, cognitive decline, chemotherapy-related cognitive decline, concussion, traumatic brain injury depression, osteoarthritis, fibromyalgia, sexual dysfunctions, CVD, ischemic heart disease (IHD), hypertensive heart disease, rheumatic heart disease (RHD) aortic aneurysms, cardiomyopathy, atrial fibrillation, congenital heart disease, endocarditis, peripheral artery disease, angina pectoris, hypertriglyceridemia, hypercholesterolemia, malnutrition or unbalanced nutrition, insufficient oral food intake, liver disease, liver dysfunction, alcoholic liver disease or renal dysfunction, comprising administering a composition of the invention.

In the context of the present invention the term “treatment” or “treating” and the like are used herein to refer to obtaining a desired pharmacological and physiological effect on the subject, including prophylactic in terms of “preventing” or partially preventing an undesired condition or symptoms from developing and/or therapeutic in terms of “curing” partial or complete curing of an already existing undesired condition. The term “treating” is used within the context of this application as treatment of subjects who are healthy and/or suffer from a disorder, disease, or impaired physiological/medical condition.

Without wishing to be hound by any theory, optimizing and/or improving and/or promoting and/or maintaining and/or reducing one or more of the various markers/factors/profiles indicated herein above and below may be beneficial for a subject administered with the composition of the invention, for example in preventing, treating or reducing the risk to develop one or more of the various disorders, diseases, conditions and indications detailed herein above or below e.g., CVD, obesity and atherosclerosis, as well as in improving cognitive abilities and other beneficial functions.

In a further aspect the invention provides a method of administering at least one water soluble choline compound to a subject comprising administering to said subject a composition of the invention, wherein upon administration of said composition the TMA or TMAO levels in one or more of the gut, urine, brain, liver, intestine or plasma of said subject are maintained or reduced.

As used herein, the term “subject” refers to a healthy subject or a subject suffering from a specific disorder or at risk of developing a specific disorder. Non limiting examples of such subjects are children born prematurely, infants born by Caesarean section, vegetarians, naturalistic and subjects with limited or deficient nutrition.

In the various aspects and embodiments of the invention, the subject may be a child. The child may be an infant or a toddler. In some embodiments the infant is one delivered by a Caesarean section. In some embodiments the infant is a newborn that may be pre-term infant and term infant. In some embodiments the subject is an infant prone to or at risk of developing a certain disorder e.g., compared to breastfed infants.

Further, the term “child” denotes infants (from day of birth, newborn, to about 12 months i.e., about 1 year) as well as toddlers (from about one year up to about the age of 3).

In some embodiments according to all aspects of the invention said subject is an infant.

An infant as used herein is meant to encompass a human infant, including hut not limited to, a newborn, a preterm and term infant, small premature infants, infants with very low birth weight (VLBW) or extreme low birth weight (ELBW) particularly those with general immaturity, for example of the gastrointestinal track or any other health risks known to a person skilled in the art. In some embodiments the infant may be one born by regular delivery, cesarean surgery (Caesarean section) as well as any other modes of delivery. The term “newborn” includes pre-mature infants, post-mature infants and full term newborns.

In some embodiments, the subject is an adult (including, a male, a female in child bearing age pre or post gestation, a teenager, an elderly senior subject). In other embodiments, the subject is a pregnant or lactating woman.

In some embodiment the subject is suffering from one or more of growth problems (overgrowth or no or insufficient growth), obesity, neurodegenerative diseases, Alzheimer's disease, Parkinson's disease, amyotrophic lateral sclerosis (ALS), dementia, stroke, cognitive decline, chemotherapy-related cognitive decline, concussion, traumatic brain injury, depression, osteoarthritis, fibromyalgia, sexual dysfunctions, CVD, ischemic heart disease (MD), hypertensive heart disease, rheumatic heart disease (RHD), aortic aneurysms, cardiomyopathy, atrial fibrillation, congenital heart disease, endocarditis, peripheral artery disease angina pectoris, hypertriglyceridemia, hypercholesterolemia, malnutrition or unbalanced nutrition, insufficient oral food intake, liver disease, liver dysfunction, alcoholic liver disease or renal dysfunction.

### DETAILED DESCRIPTION OF EMBODIMENTS

### EXAMPLE 1

### Production of Compositions According To The Invention

### A. Extraction and Concentration of Water Soluble Choline Compounds Using Ethanol as a Solvent to Obtain a Powder Product.

Whey stream from dairy production was concentrated by evaporation of water and then crystalized to yield lactose crystals that were separated by filtration from their mother liquor. Mother liquor was partially demineralized by nanofiltration membrane and dried by means of spray drying to obtain dry powder.

Ten grams of the dried powder were mixed with 200 ml of ethanol 90% (with 10% water) for 2 hours at 40 ° C. to obtain slurry. The slurry was filtered through Buchner filter in order to separate between the filtrate and the solids. The filtrate was evaporated, under reduced pressure, using rotary evaporator to obtain dry powder. The dry powder obtained contained 5.5% GPC and 0.52% PCh (as determined by ³¹P-NMR).

### B. GPC Enrichment Using Ion Exchange Columns.

Ten grams of the final product from Example 1A (containing 5.5% GPC and 0.52% PCh) were dissolved in 40 ml of water and then were passed through a glass column containing 100 ml of strong anionic exchange resin (Doc2001). The solution coming out of the first column was then transferred through a strong cationic resin (001×7) column and was neutralized by a weak anionic resin (D301) column, in order to remove minerals and to obtain a neutralized filtrate. All resins were obtained from JIANGSU SUQING WATER TREATMENT ENGINEERING GROUP CO. Finally, the neutralized filtrate was evaporated using a rotary evaporator, under reduced pressure, to receive a product containing 20.5% GPC and 0.03% of PCh (as determined by ³¹P-NMR).

### C. GPC Enrichment Using a Silica Chromatography Procedure

Ten grams of the final product of Example 1A (containing 5.5% GPC) were dissolved in 20 ml of ethanol:water (80%:20% v/v) solution and were loaded on a silica gel chromatography column filled with 70 grams of Davisil “LC60A 20-45 μm” silica from the Grace Company. 500 ml ethanol:water (80%:20% v/v) was then transferred through the column in order to separate a lactose containing fraction from a GPC containing fraction. After the first 200 ml of the ethanol containing solvent was applied to the column, the collection of GPC containing fractions of the filtrate commenced. The GPC containing fractions were then evaporated using rotary evaporator, under reduced pressure, to obtain a dry powder, The dried powder obtained was injected to the HPLC with ELS detector against a sample of the product of Example 1A, in the same concentration. The relative peak area of the GPC was about 5 fold higher in the purified product compared to the product of example 1A.

### D. Purification of Choline Compounds From Whey Stream With Methanol as a Solvent to Obtain a Powder Product.

Whey stream from dairy production was used to produce whey protein concentrate by diafiltration using Ultra filtration membranes. The permeate from the membranes was demineralized by means of electrodialysis. The mineral free stream was dried by spray dryer to produce a powder. Five grams of this dried powder were mixed with 40 ml of methanol for 2 hours at 25° C. The whole sample was then centrifuged for 5 minutes at 6,000 RPM in order to separate between the solution and the solids. The solution was evaporated using rotary evaporator, under reduced pressure, to receive dry powder. The dry powder obtained contained 7.6% GPC and 0.4% PCh (By ³¹P-NMR).

### E. Purification of GPC From “Lactosalt Optitase”

Dairy salts fraction called “Lactosalt Optitase” (Armor) containing about 85% salts, 5% moisture and 0.5% protein was purified by electrodialysis. Purification was performed using a PCCell ED 64-4 Electrodialysis cell unit. This unit has a 10 parallel cell pair stack structure. The active size of each membrane is 8×8 cm (active area of 0.0064 m2). Hence, total active area is 0.064 m².

For the electrolyte circuit, a 0.25 M solution of sodium sulfate was used. The anolyte and the catholyte chambers were connected in series. A circulating NaCl solution served as the concentrate. Its initial concentration was around 1000 mg/lit. 1 liter of solution containing 10 gr of “Lactosalt Optitase” dissolved in demineralized water was fed to the circulating chamber. The voltage was pre-set at its highest value (36.5 Volts for the stack). Recirculation was stopped when further significant decreases in conductivity were no longer noted in the salts solution. Sample of the purified solution was dried by rotary evaporator, under reduced pressure, to dryness. The dried product obtained was injected to the HPLC with ELS detector against a sample of the same concentration of the original “Lactosalt Optitase”. The relative peak area of the GPC was about 10 fold higher in the purified product compared to the raw material.

### F. Purifeation of GPC From Krill Meal

200 gr. of Superba Krill meal were mixed together with 1 liter of methanol for one hour at 25° C. The whole solution was then filtered through Buchner filter in order to separate between the filtrate and the solids. The filtrate was evaporated using rotary evaporator to obtain 35 gr of oil. Thirty five ml of purified water and thirty five ml of butanol were added to the oil and the solution was mixed for few minutes. Phase separation was achieved in a separatory funnel. The bottom phase was evaporated to obtain 2.78 gr. which contained 7.1% GPC (by ³¹P-NMR).

### G. Preparation of Gummy Bears Containing GPC

0.4 gr of Citric acid and 0.37 gr of Trisodium citrate were dissolved in 30 ml water using agitation. The solution was heated up to 75° C., followed by the addition of 5 gr of white sugar (Sucrose) and 1.5 gr of Citrus Pectin. The mixture was heated up to 100° C., and agitated at 100° C. for 2-3 minutes. 30 gr of glucose syrup 80% and 50 gr white sugar (Sucrose) were added and the mixture was heated up to 108° C. under continuous agitation until full dissolution and 78° Bx is achieved (about 40-50 minutes). The solution was cooled down to 1.00° C. and 1.06 gr product of example No. 1A were added. Agitation was continued at 100° C. for 2-3 minutes, and the following flavorings and colorings were added: 0.5 gr Adipic acid, 0.15 gr strawberry flavor essence, 0.5 ml Lemon juice, 0.15 gr shade ruby red essence, 0.77 gr Citric acid 50%. Agitation was continued at 100° C. for 2-3 more minutes, than heat source was stopped and product obtained was poured into molds at 90-100° C. Molds were placed in an air conditioned room for about 48 hours for drying.

### EXAMPLE 2

### GPC Stability Of Natural Vs. Synthetic Choline Composition in Stomach Model

More than 90% of orally administrated GPC is absorbed from the intestine. Once absorbed, GPC is rapidly circulated to all organs and taken up into the cells. It is thus desirable that GPC will be minimally affected by gastrointestinal conditions and remain intact without any modifications which might affect its activity and efficacy.

In order to test the gastrointestinal stability of natural (mammalian milk derived) water soluble choline compounds according to the invention, in comparison with synthetic, conventional choline compounds an in vitro gastric (stomach) model was used.

In vitro gastric model was conducted as previously described by Kanner and Lapidot 2001 using simulated gastric fluid (SGF) as an artificial dissolution medium intended to represent stomach conditions. SGF was prepared according to the U.S. Pharmacopoeia by dissolving 0.2% w/w sodium chloride and 0.32% w/w of purified pepsin (derived from porcine stomach mucosa) in acidic water, pH about 1.2.

Choline compounds were purified from mother liquor from lactose crystallization. The purification included two stages: first stage of membrane purification and a second stage of crystallization.

Synthetic water soluble choline compound was produced from soy lecithin by a reaction using Sodium methoxide as a catalyst to obtain GPC and methyl esters. Several purification steps were performed in order to separate between the methyl esters and GPC. In addition, a mineral removal step was performed using ion exchangers.

Both natural and synthetic water soluble choline compounds (compositions are detailed in Table 2) were incubated with SGF at 37° C. in a shaking bath for 180 minutes and then the samples were analyzed for GPC levels by HPLC.

The results, presented in Table 2, show that a composition containing about 5% w/w of natural, water soluble, choline compound from dairy source did not demonstrate any GPC degradation (GPC level remained constant) following 180 minutes incubation in gastric model conditions. On the other hand, the composition which contained synthetic, water soluble, choline compound, demonstrated degradation of about 5.4% of the GPC (from 30.50 mg GPC to 28.84 mg GPC).

These results demonstrate that compositions containing water soluble choline compounds according to the invention are more stable in gastric conditions than other compositions which contain conventional choline compounds.

TABLE 2

| | Composition | | | |
|---|---|---|---|---|
| | A | | B | |
| Source of water | Natural (mammalian | | Synthetic | |
| soluble choline | source) | | | |
| compound | | | | |
| % PCh and Choline | 12.5% | | <1% | |
| from total choline | | | | |
| compounds (w/w) at | | | | |
| baseline | | | | |
| | | after | | after |
| | at time 0 | 180 min | at time 0 | 180 min |
| GPC (mg GPC/ml | 35.35 | 35.48 | 30.50 | 28.84 |
| SGF) | | | | |

| | | |
|---|---|---|
| % GPC Degradation | 0 | 5.4 |

### EXAMPLE 3

### The Effect of Different Water Soluble Choline Compounds on Factors Related to Infant Growth

Study Design

The ability of different water soluble choline compounds to affect blood levels of factors related to infant growth was investigated in an animal model of neonatal Sprague Dawley rats aged 3-5 days.

Animals were randomly assigned to one of three diets, twelve rats per group.

Animals within a litter were randomly assigned across treatments.

The study groups were:

Group A: Rats fed with formula containing choline

Choline chloride was purchased from Sigma Chemical Company.

Group B. Rats fed with formula containing GPC and phosphocholine (wherein Phosphocholine>GPC). Synthetic water soluble choline compound was produced from soy lecithin by a reaction using sodium methoxide as a catalyst to obtain GPC and methyl esters. Several purification steps were performed in order to separate between the methyl esters and GPC. In addition, a mineral removal step was performed using ion exchangers. Phosphocholine chloride calcium salt tetrahydrate was purchased from Sigma Chemical Company.

Group C: Rats fed with formula containing GPC (wherein GPC>Phosphocholine) from bovine milk natural source. Choline compounds were purified from mother liquor from lactose crystallization. The purification included two stages: Ion exchange purification using a strong cationic resin (001×7) column and weak anionic resin (D301) column (all resins were obtained from JIANGSU SUQING WATER TREATMENT ENGINEERING GROUP CO.) and chromatographic purification based on UBK535K resin from Diaion Company.

All formula-based diets contained the same added choline equivalent levels that originated from different water soluble choline compounds. The water soluble choline compound composition of each diet is detailed in Table 3.

TABLE 3

| | Group A | Group B | Group C |
|---|---|---|---|
| Source of the choline compound | synthetic | synthetic | natural |
| Total choline equivalent (mg/liter | 679 | 680 | 679 |
| formula) | | | |
| Choline (mg/liter formula) | 678.4 | NA | NA |
| Choline (μM) | 6525 | NA | NA |
| GPC (mg/liter formula) | NA | 826.65 | 1676 |
| GPC (μM) | NA | 3217 | 6525 |
| Phosphocholine (mg/liter | NA | 610.4 | 1.35 |
| formula) | | | |
| Phosphocholine (μM) | NA | 3317 | 2 |
| GPC out of composition (% w/w) | NA | 39.7 | 24.8 |
| GPC out of choline compound | NA | 57.5 | 99 |
| (% w/w) | | | |
| Choline compound out of | 100 | 69 | 24.8 |
| composition (% w/w) | | | |
| Choline + phosphocholine | 100 | 41.9 | <1 |
| compound out of choline | | | |
| compound composition (% w/w) | | | |

NA = Not Applicable

Gastrostomy tube fed infant rats: The gastrostomy tube fed rat pup is model to mimic infants fed formula, using tube feeding to overcome the difficulties in bottle feeding of neonatal rats. The formulas were prepared to resemble the composition of rat milk with ingredients modified to meet the study objectives, The model enables complete control of the volume and thus nutrient intake. This avoids any difficulties due to variable intake across treatment groups. The animals were reared by milk feeding from 3-5 to 18-20 days of age. Formula volume which was administered to the rats was calculated daily based on the animal weight.

Plasma and Tissue collection: Blood samples were centrifuged at 2000 g×10 minutes, and plasma was recovered. In order to standardize tissue sample location from every animal, tissue samples harvest was done following the same protocol.

List of Analyses:

In plasma: cholesterol (total, VLDL+LDL, HDL), triglycerides, phosphatidycholine, free choline, glycerophosphocholine, phosphocholine, betaine, dimethylglycine (DMG), homocysteine, methionine, cysteine, Trimethylamine (TMA) and Trimethylamine N-oxide (TMAO), Ketone bodies, Folate, S-adenosyl methionine (SAM), S-adenosyl homocysteine (SAH), Growth hormone, Insulin-like growth factor 1 (IGF-1), IGF 3 binding protein (IGF3BP) and ferritin.

In urine: TMA and TMAO.

In liver: SAM, SAH and triglycerides.

Results

Plasma and urine samples were analyzed for levels of various lipids and metabolites and tissue samples were weighted. The results are presented in Table 4.

Plasma parameters: plasma levels of choline in groups B and C were higher in comparison with the choline levels in group A. Choline levels in group C were higher also in comparison with group B. Choline/betaine ratio and IGF-I levels increased in groups B and C in comparison with group A. Triglyceride and cholesterol levels were higher in group C in comparison with both group A and group B and better resembled the high triglyceride and cholesterol levels of suckling rats (128.4±1111.3 mg/dl and 158.5±36.0 mg/dl, respectively). TMAO levels in group B and C were lower in comparison with those of group A. Plasma SAM and SAH levels were higher in group C in comparison with both group A and B. Folate levels were lower in group C in comparison with groups A and B.

Urine parameters: TMA and TMAO levels in the urine of group B and C animals reduced in comparison with those of group A.

Tissue parameters: body and liver weights were lower in group C in comparison with those of group A and B.

TABLE 4

| | | |
|---|---|---|
| Group A | Group B | Group C |

Plasma parameters

| | | | |
|---|---|---|---|
| Choline (μM) | 11.5 ± 3.5 | 12 ± 3.4 | 13.7 ± 4.0 |
| Betaine (μM) | 151.3 ± 43.2 | 122.4 ± 31.9 | 148.5 ± 30.9 |
| DMG (μM) | 18.0 ± 3.8 | 17.6 ± 5.1 | 15.6 ± 1.9 |
| Ketones (mM) | 0.5 ± 0.2 | 0.4 ± 0.2 | 0.4 ± 0.2 |
| Triglycerides (mg/dl) | 99.5 ± 55.7 | 92.1 ± 45.5 | 127.9 ± 86.6 |
| Cholesterol (total) | 77.1 ± 20.9 | 88.5 ± 10.2 | 89.0 ± 17.7 |
| (mg/dl) | | | |
| TMA (μM) | 0.2 ± 0.1 | 0.1 ± 0.1 | 0.2 ± 0.1 |
| TMAO (μM) | 1.2 ± 0.4 | 0.9 ± 0.4 | 1.0 ± 0.3 |
| IGF-I (ng/ml) | 184.9 ± 51.1 | 235.7 ± 56.2 | 203.4 ± 44.2 |
| Plasma SAM (nM) | 448.6 ± 24.6 | 404.5 ± 37.2 | 486.3 ± 15.9 |
| Plasma SAH (nM) | 123.5 ± 31.9 | 127.3 ± 70.8 | 150.4 ± 24.5 |
| Folate (nM) | 128.5 ± 18.3 | 118.7 ± 26.6 | 115.6 ± 9.6 |
| Homocysteine (μM) | ND | ND | ND |
| Methionine (μM) | ND | ND | ND |
| Cysteine (μM) | ND | ND | ND |
| HDL (mg/dL) | ND | ND | ND |
| GPC (μM) | ND | ND | ND |
| Phosphatidylcholine | ND | ND | ND |
| and lyso | | | |
| phosphatidylcholine | | | |
| (μM) | | | |
| IGF3BP (mg/l) | ND | ND | ND |
| Ferritin (ng/ml) | ND | ND | ND |

Urine parameters

| | | | |
|---|---|---|---|
| TMA (μM) | 8.0 ± 9.6 | 2.4 ± 1.4 | 4.6 ± 3.1 |
| TMAO (μM) | 158.3 ± 79.0 | 73.3 ± 54.0 | 139.1 ± 89.0 |

Tissue parameters

| | | | |
|---|---|---|---|
| Body weight (gr) | 41.3 ± 5.9 | 48.2 ± 5.8 | 39.7 ± 5.1 |
| Liver weight (gr) | 1.7 ± 0.4 | 2.1 ± 0.3 | 1.6 ± 0.3 |
| Liver weight/100 gr | 4.1 ± 0.5 | 4.3 ± 0.3 | 3.9 ± 0.3 |
| body weight | | | |
| Liver SAM (μM) | 120.3 ± 6.1 | 114.1 ± 5.3 | 115.4 ± 7.6 |
| Liver SAH (μM) | 18.9 ± 0.9 | 17.5 ± 1.0 | 17.4 ± 1.1 |

*results are given as mean ± SD,
ND = Not Determined

### CONCLUSION

The above results demonstrate that animals in group C which were fed with formula containing choline compound derived from mammalian milk (bovine milk) demonstrated higher choline bioavailability, better lipid profile and a reduced body and liver weight in comparison with both groups A and B in which rats were fed with formula containing synthetic choline compounds. Furthermore, group C demonstrated plasma SAM and SAH increase and folate reduction in comparison with groups A and B. To addition, both groups B and C demonstrated an increase in plasma choline/betaine ratio and IGF-1 levels and a reduced TMA and TMAO levels in comparison with group A.

The improved choline bioavailability, improved plasma and urine profile and the reduced body and liver weight which were observed with group C (and to a lesser extent in group B) may be indicative of healthy growth of the animals and be associated with for example optimal growth and statural growth of infants. They may also be associated with lower risk to develop various diseases such as CVD, obesity and atherosclerosis and other beneficial functions.

### Example 4

### GPC Stability in Infant Formula Containing Water Soluble Choline Compounds According to the Invention in Comparison With Infant Formula Containing Conventional Water Soluble Choline Compounds.

Two types of infant formulas were prepared in pilot scale by the following method: Skimmed milk powder, lactose and concentrated whey protein (80%) were mixed into distilled water by a high speed agitator and warmed to 65-70° C. Following 5 minutes of mixing, different recipes of waters soluble choline compounds, minerals, nucleotides, amino acids and vitamins were added. Following an additional 15 minutes, an oil mixture containing vegetable oils including ARA (Arachidonic acid) oil and DHA (Docosahexaenoic acid) oil were added. Mixing continued for additional 15 minutes. Then, the mixture was homogenized by “APV Rannie pressure homogenizer” with two-stage assembly: 70 Bars at stage 1 and 240 Bars at stage 2. Next, the homogenized mixture was spray dried by typical “Spray Dryer” at a rate of 20 liter/hr with air inlet temp of about 180° C. and air outlet temp of about 80° C. Dried powder was collected and dry blended with a premix (about 0.37%) of minerals and elemental substances.

At the end of this stage the two formulas contained a different composition of choline compounds: while 348-80-1 contained a molar ratio of GPC>PCh, formula no. 348-80-6 contained a molar ratio of GPC<PCh.

100 gr aliquots from each formula were packed under nitrogen environment in sealed airtight aluminum packages and stored at 25° C.±2 (Humidity 60%±5%) in temperature and moisture controlled storage chambers.

At baseline and following 12 months of storage, samples were analyzed for GPC and PCh content.

Table 5 shows stability results following 12 months at 25° C.±2 (Humidity 60%±5%). Sample 348-80-6, containing PCh>GPC ratio demonstrated minor GPC degradation level (about 4%) while formula 348-80-1, containing GPC>PCh ratio, resulted in about 38% GPC degradation. These results demonstrate that compositions containing water soluble choline compounds according to the invention are more stable than other compositions which contain conventional choline compounds.

TABLE 5

| | Formula | |
|---|---|---|
| | 348-80-1 | 348-80-6 |
| GPC-Phosphocholine Ratio | GPC > | GPC < |
| | Phosphocholine | Phosphocholine |
| % w/w PCh and choline from | >1 | >1 |
| water soluble choline | | |
| compounds | | |
| % w/w Choline | 0.09 | 0.013 |
| % w/w PCh T0 | NA | 0.09 |
| % w/w PCh Following 12 | NA | 0.09 |
| month of storage at 25° C. ± 2 | | |
| % w/w GPC T0 | 0.037 | 0.024 |
| % w/w GPC Following 12 | 0.023 | 0.023 |
| month of storage at 25° C. ± 2 | | |
| % GPC degradation | 38 | 4.2 |
| Following 12 month of storage | | |
| at 25° C. ± 2 | | |
| % PCh degradation | NA | 0 |
| Following 12 month of storage | | |
| at 25° C. ± 2 | | |

NA = Not Applicable,
T0 = baseline values

1-28. (canceled)

29. A method for one or more of:
(i) optimizing, improving, promoting, increasing or maintaining plasma levels of one or more of ferritin, iron, growth hormone, insulin-like growth factor, insulin-like growth factor binding protein or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same,
said method comprising administering to said subject a composition comprising at least one water soluble choline compound selected from a group consisting of GPC, PCh and choline; wherein said at least one water soluble choline compound is derived from mammalian milk, and wherein the concentration of said at least one water soluble choline compound is at least 0.5% w/w of the composition.

30. A method for one or more of:
(i) optimizing, improving, promoting, increasing or maintaining plasma levels of one or more of ferritin, iron, growth hormone, insulin-like growth factor, insulin-like growth factor binding protein or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same,
said method comprising administering to said subject a composition comprising at least one water soluble choline compound selected from a group consisting of GPC, PCh and choline; wherein said at least one water soluble choline compound is derived from at least one natural source, and wherein the concentration of said at least one water soluble choline compound is at least 0.5% w/w of the composition.

31. The method according to claim 29 wherein PCh comprises at least 0.02% w/w of the composition, and wherein GPC comprises at least 0.5% w/w of the composition.

32. The method according to claim 29, wherein GPC comprises between about 20% w/w to about 100% w/w of the at least one water soluble choline compound, and PCh and choline comprise between about 1% w/w to about 60% w/w of at least one water soluble choline compound.

33. The method according to claim 29 wherein the composition comprises at most 50 ppm of at least one of TMA, TMAO, ethylene oxide, glycidol or any combination of the same.

34. A method for one or more of:
(i) optimizing, improving, promoting, increasing or maintaining plasma levels of one or more of ferritin, iron, growth hormone, insulin-like growth factor, insulin-like growth factor binding protein or ketone bodies in a subject;
(ii) optimizing, improving, promoting, increasing or maintaining one or more of plasma lipid profile, plasma triglyceride levels and plasma cholesterol levels in a subject;
(iii) promoting optimal growth of a subject, improving statural growth of a subject or a combination of the same; and
(iv) preventing, delaying or reducing subject's obesity, reducing subject's body mass index or a combination of the same,
said method comprising administering to said subject a composition comprising at least two water soluble choline compounds selected from a group consisting of GPC, PCh and choline; wherein said at least two water soluble choline compounds comprise at least 0.5% w/w of the composition; and wherein PCh and choline together comprise at least 1% w/w of said water soluble choline compounds.

35. The method according to claim 34, wherein PCh comprises between about 1% w/w to 70% w/w of said water soluble choline compounds.

36. The method according to claim 34, wherein said at least two water soluble choline compounds comprise at least 1% w/w of the composition.

37. The method according to claim 34, comprising GPC, PCh and choline.

38. The method according to claim 34, wherein said GPC comprises between 20% w/w to about 100% w/w of said water soluble choline compounds.

39. The method according to claim 34, wherein said PCh comprises between 1% w/w to about 70% w/w of said water soluble choline compounds.

40. The method according to claim 34, wherein choline comprises between about 1% w/w to 25% w/w of said water soluble choline compounds.

41. The method according to claim 34, wherein at least one of said GPC, PCh or choline is derived from a natural source.

42. The method according to claim 34, wherein at least one of said GPC, PCh or choline is derived from mammalian milk

43. The method according to claim 34, wherein the molar concentration of PCh is greater than the molar concentration of GPC.

44. The method according to claim 29, wherein said composition is a pharmaceutical, a dietary supplement, a medical food or a nutraceutical composition.

45. The method according to claim 29, wherein the subject is an infant.
